(19) United States
(12) Patent Application Publication
(10) Pub. No.: US 2023/0055713 A1
(43) Pub. Date: Feb. 23, 2023

(54) COMPOSITION FOR STRENGTHENING SKIN BARRIER OR MOISTURIZING SKIN COMPRISING TILIANIN

(71) Applicants: COSMAX NBT, INC., Seongnam-si (KR); COSMAX NS, INC., Seongnam-si (KR)

(72) Inventors: Jin Hee YOO, Seoul (KR); Su Young CHOI, Yongin-si (KR); Mann-Seok YUN, Yongin-si (KR); Won Il YUN, Seoul (KR)

(73) Assignees: COSMAX NBT, INC., Seongnam-si (KR); COSMAX NS, INC., Seongnam-si, (KR)

(21) Appl. No.: 17/573,783

(22) Filed: Jan. 12, 2022

Related U.S. Application Data
(60) Provisional application No. 63/227,543, filed on Jul. 30, 2021.

(51) Int. Cl. A61K 36/532; A23L 33/105; A61K 8/9789; A61Q 19/00

(52) U.S. Cl. CPC A61K 36/532; A23L 33/105; A61K 8/9789; A61Q 19/00

(57) ABSTRACT

A composition for strengthening a skin barrier or moisturizing skin is provided. The composition includes tilianin. The composition may be a food, a health functional food composition, a quasi-drug composition and/or a composition for external use on the skin. The composition increases the contents of moisture and hyaluronic acid in the skin and exhibits an upregulating effect on HAS. A method for strengthening a skin barrier or enhancing skin moisture using tilianin, and provides a use of tilianin in the preparation of food or medicine for strengthening a skin barrier or enhancing skin moisture are also provided.

### CROSS REFERENCE TO RELATED APPLICATIONS

This application claims the benefit of and priority to U.S. Provisional Application No. 63/227,543 filed Jul. 30, 2021, the content of which is incorporated by reference in its entirety.

### SEQUENCE LISTING

The content of the electronically submitted sequence listing, file name: Sequence_Listing_As_Filed.txt; size: 1,801 bytes; and date of creation: Jan. 10, 2022, filed herewith, is incorporated herein by reference in its entirety.

### TECHNICAL FIELD

The present invention relates to a composition for strengthening a skin barrier or moisturizing skin including tilianin, and more specifically, the present invention provides a food composition, a health functional food composition, a quasi-drug composition, a cosmetic composition and/or a composition for external use on the skin, using tilianin which increases the contents of moisture and hyaluronic acid in the skin and exhibits an upregulating effect on HAS. In addition, the present invention provides a method for strengthening a skin barrier or enhancing skin moisture using tilianin, and provides a use of tilianin in the preparation of food or medicine for strengthening a skin barrier or enhancing skin moisture. Furthermore, based on the skin harrier strengthening and skin moisturizing effects of tilianin, the present invention provides a pharmaceutical composition, a health functional food composition, a cosmetic composition and/or a composition for external use on the skin for preventing, ameliorating or treating skin disease caused by damage to a skin barrier or dry skin using tilianin, additionally provides a method for preventing, ameliorating or treating skin disease caused by damage to a skin barrier or dry skin using tilianin, and provides a use of tilianin in the preparation of food or medicine for preventing, ameliorating or treating skin disease caused by dry skin.

### BACKGROUND ART

Skin plays a very important role as a barrier function to protect individuals from the outside. The barrier function is a function of defending against various stimuli from the outside (chemicals, air pollutants, dry environment, ultraviolet radiation, etc.) and preventing excessive diffusion of water in the body through the skin, and this protective function can be maintained only when the stratum comeum composed of keratinocytes is normally formed.

Among the epidermis, the outermost stratum corneum is formed from keratinocytes and is composed of keratinocytes with complete differentiation and a lipid layer surrounding the same. Keratinocytes are characteristic cells in which basal cells that continuously proliferate in the lowermost layer of the epidermis have undergone stepwise morphological and functional changes and rise to the skin surface. After a certain period of time, old keratinocytes are removed from the skin and new keratinocytes take over their functions, and this repetitive sequence of changes is called “differentiation of epidermal cells” or “keratinization”. During the keratinization process, keratinocytes form the stratum comeum while producing natural moisturizing factors (NMF) and intercellular lipids (ceramide, cholesterol and fatty acids) and make the stratum comeum robust and flexible, thereby retaining the function as a skin barrier. When the stratum corneum becomes thin and weakened, the structures of the keratin and the lipid membrane of the cell membrane, which are tightly bound like bricks and cement, become loose and form a gap, and eventually the moisture is evaporated to thy and the protective function against harmful substances is weakened.

The stratum corneum can easily lose its function due to lifestyle factors such as excessive washing, bathing and the like, environmental factors such as drying, air pollutants and the like, and endogenous diseases such as atopic skin, senile skin and the like. In fact, due to a variety of factors that have become increasingly popular in modem practice, there is an increasingly growing trend in recent years for people complaining dry skin symptoms and the resulting skin disorder. Therefore, various studies have been conducted to supply moisture from the outside to maintain adequate skin moisture, or to prevent the loss of moisture from the body, and in practice, various moisturizers with the water retaining ability have been developed. As skin moisturizers, it is common to use substances that can increase water retention in the stratum comeum, such as lipid components such as ceramides and the like, and essential fatty acids, lipid complexes and the like.

However, in recent years, the number of harmful factors to the skin is gradually increasing, the generation and exfoliation rates of the stratum comeum are slowed down due to changes in dietary habits, and the amount of moisturizing factors and lipids in the stratum corneum are decreased due to a decrease in the function of keratinocytes such that the number of people with skin in which the stratum corneum does not function as a normal skin barrier is increasing. Such damage to the skin harrier function causes various diseases such as dry skin, atopic dermatitis, contact dermatitis, psoriasis and the like. In order to cure such diseases, in addition to conventional skin moisturizers, it is necessary to develop functional products that strengthen a skin barrier or enhance the moisturizing effect as food-type skin nutrients.

Meanwhile, although Korean Registered Patent No. 10-1805801 discloses a pharmaceutical composition for preventing or treating Parkinson's disease including tilianin as an active ingredient, the effects of tilianin for strengthening a skin barrier and/or moisturizing skin have not been revealed to date.

### DISCLOSURE

### Technical Problem

Under these circumstances, the inventors of the present invention have confirmed that tilianin exhibits excellent skin barrier strengthening and/or skin moisturizing effects by restoring the reduced contents of moisture and hyaluronic acid in the skin due to UVB irradiation and upregulating the mRNA expression of HAS, and thereby completed the present invention.

Accordingly, it is an object of the present invention to provide a composition for strengthening a skin barrier or moisturizing skin using tilianin.

It is another object of the present invention to provide a method for strengthening a skin barrier or enhancing skin moisture using tilianin.

It is still another object of the present invention to provide a use of tilianin in the preparation of food or medicine for strengthening a skin barrier or enhancing skin moisture.

In another aspect, it is an object of the present invention to provide a composition for preventing, ameliorating or treating skin disease using tilianin.

It is another object of the present invention to provide a method for preventing, ameliorating or treating skin disease using tilianin.

It is still another object of the present invention to provide a use of tilianin in the preparation of food or medicine for preventing, ameliorating or treating skin disease.

### Technical Solution

In order to solve the aforementioned problems, the present invention provides a food composition for strengthening a skin barrier or moisturizing skin, including tilianin or a sitologically acceptable salt thereof.

Additionally, the present invention provides a health functional food composition for moisturizing skin, including tilianin or a sitologically acceptable salt thereof.

In addition, the present invention provides a quasi-drug composition for strengthening a skin barrier or moisturizing skin, including tilianin or a pharmaceutically acceptable salt thereof.

In addition, the present invention provides a cosmetic composition for strengthening a skin barrier or moisturizing skin, including tilianin or a cosmetically acceptable salt thereof.

In addition, the present invention provides a composition for external use on skin for strengthening a skin barrier or moisturizing skin, including tilianin or a pharmaceutically acceptable salt thereof.

In another aspect, the present invention provides a method for strengthening a skin barrier or enhancing skin moisture, including administering a composition including tilianin, a sitologically acceptable salt thereof or a pharmaceutically acceptable salt thereof to a subject in need thereof.

Additionally, the present invention provides a use of tilianin, a sitologically acceptable salt thereof or a pharmaceutically acceptable salt thereof in the preparation of food or medicine for strengthening a skin barrier or enhancing skin moisture.

According to a preferred exemplary embodiment of the present invention, the tilianin may be derived from an Agastache rugosa extract.

According to another preferred exemplary embodiment of the present invention, the tilianin, sitologically acceptable salt thereof or pharmaceutically acceptable salt thereof may be included at 0.0001 to 20 wt. % based on the total weight of the composition.

According to still another preferred exemplary embodiment of the present invention, the tilianin may exhibit an effect of any one or more of the following a) to d):

a) increasing the content of moisture in the skin;

b) increasing the content of hyaluronic acid in the skin;

c) promoting the synthesis of hyaluronic acid; and

d) reducing transepidermal water loss.

In still another aspect, the present invention provides a pharmaceutical composition for preventing or treating skin disease, including tilianin or a pharmaceutically acceptable salt thereof.

In addition, the present invention provides a health functional food composition for preventing or ameliorating skin disease, including tilianin or a sitologically acceptable salt thereof.

In addition, the present invention provides a composition for external use on skin for preventing or treating skin disease, including tilianin or a pharmaceutically acceptable salt thereof.

In addition, the present invention provides a cosmetic composition for preventing or ameliorating skin disease, including tilianin or a cosmetically acceptable salt thereof.

Additionally, the present invention provides a method for preventing, ameliorating or treating skin disease, including administering tilianin, a sitologically acceptable salt thereof or a pharmaceutically acceptable salt thereof to a subject in need thereof.

In addition, the present invention provides a use of tilianin, a sitologically acceptable salt thereof or a pharmaceutically acceptable salt thereof in the preparation of food or medicine for preventing, ameliorating or treating skin disease.

According to a preferred exemplary embodiment of the present invention, the tilianin may be derived from an Agastache rugosa extract.

According to another preferred exemplary embodiment of the present invention, the skin disease may be any one or more selected from the group consisting of dry skin, eczema, psoriasis, atopic dermatitis, ichthyosis and pemphigus.

According to still another preferred exemplary embodiment of the present invention, the tilianin or a sitologically acceptable salt thereof may exhibit an effect of any one or more of the following a) to d):

a) increasing the content of moisture in the skin;

b) increasing the content of hyaluronic acid in the skin;

c) promoting the synthesis of hyaluronic acid; and

d) reducing transepidermal water loss.

### Advantageous Effects

The composition for strengthening a skin barrier or moisturizing skin using tilianin according to the present invention exhibits excellent skin barrier strengthening and/or skin moisturizing effects by restoring the reduced contents of moisture and hyaluronic acid in the skin due to ultraviolet rays, upregulating HAS, and reducing the degree of transepidermal water loss (amount of transepidermal water loss). Through these effects, it can be utilized to prevent, ameliorate or treat various skin diseases caused by damage to a skin barrier or dry skin. In addition, as the skin barrier strengthening and/or skin moisturizing effects by oral administration of tilianin were confirmed, the effects can be further enhanced by formulating as an ingestible skin nutrient or the like and in combination with other known moisturizers.

### BRIEF DESCRIPTION OF DRAWINGS

FIG. 1A is a graph showing the effects of an Agastache rugosa extract (ARE) and tilianin on skin hydration, and FIG. 1B is a graph showing the effects of an Agastache rugosa extract (ARE) and tilianin on the degree of transepidermal water loss (TEWL). Each result value was expressed as mean±SD (^{##}p<0.01 vs. CON group; **p<0.01 vs. UVB group).

FIG. 2 is a graph showing the effects of an Agastache rugosa extract (ARE) and tilianin on the hyaluronic acid level (^{##}p<0.01 vs. CON group; **p<0.01 vs. UVB group).

FIG. 3 is a graph showing the effects of an Agastache rugosa extract (ARE) and tilianin on the expression of hyaluronic acid synthase. Each result value was expressed as mean±SD (^{#}p<0.05, ^{##}p<0.01 vs. CON group; *p<0.05 and **p<0.01 vs. UVB group).

### MODES OF EMBODIMENTS

As described above, in spite of the development of various skin moisturizers, due to an increase in harmful factors to the skin, changes in dietary patterns and the like, there is a need to develop functional products that exhibit an effect of strengthening a skin barrier or enhancing moisture retention as food-type skin nutrients as well as skin moisturizers.

As such, the inventors of the present invention have confirmed that tilianin exhibits excellent skin barrier strengthening and/or skin moisturizing effects by restoring the contents of moisture and hyaluronic acid in the skin reduced due to ultraviolet rays and upregulating HAS, and have sought to solve the above-mentioned problems by providing a composition for strengthening a skin barrier and/or moisturizing skin using tilianin.

Accordingly, a first aspect of the present invention relates to a food composition including tilianin or a sitologically acceptable salt thereof and/or a health functional food composition including tilianin or a sitologically acceptable salt thereof

In the food composition of the present invention, tilianin which is included as an active ingredient is a compound having the structure of [Chemical Formula 1] below, and it has various physiological effects such as anti-hypertensive, anti-diabetic and anti-oxidative effects (Garcia-Diaz J A et al., (2016) Antidiabetic, antihyperlipidemic and anti-inflammatory effects of tilianin in streptozotocin-nicotinamide diabetic rats. Biomed. Pharmacother., 83: 667-675; Hemandez-Abreu et al., (2009) Antihypertensive and vasorelaxant effects of tilianin isolated from Agasiache mexicana are mediated by NO/cGMP pathway and potassium channel opening. Biochem. Pharmacol., 78(1): 54-61). In addition, it has been shown to exhibit an anti-inflammatory effect through inhibition of NF-κB expression (Nam et al., (2005) Inhibition of cytokine-induced IκB kinase activation as a mechanism contributing to the anti-atherogenic activity of tilianin in hyperlipidemic mice. Atherosclerosis, 180(1): 27-35).

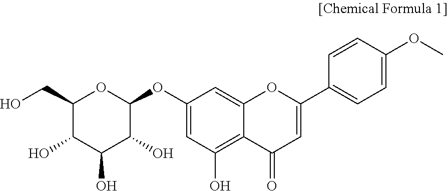 [SMILES: COc1ccc(-c2cc(=O)c3c(O)cc(O[C@@H]4O[C@H](CO)[C@@H](O)[C@H](O)[C@H]4O)cc3o2)cc1]

In the food composition of the present invention, the tilianin may be synthesized artificially or may be commercially obtained and used. In addition, it may be used after purification from a substance containing tilianin, a natural source or the like. For example, the tilianin may be derived from an Agastache rugosa extract, but is not limited thereto.

The use of the term “purified” is intended to mean that tilianin is in a concentrated form compared to a form obtainable from natural origin. The purified components may be concentrated from the natural sources thereof or obtained by chemical synthesis methods.

In the food composition of the present invention, the tilianin or sitologically acceptable salt thereof may be included at 0.0001 to 20 wt. % based on the total weight of the composition, but is not limited thereto. For example, in the food composition of the present invention, the tilianin or sitologically acceptable salt thereof may be included without limitation at an amount capable of inducing a desired effect by being administered to the body, that is, an amount capable of inducing the effects of a) increasing the content of moisture in the skin; b) increasing the content of hyaluronic acid in the skin; c) promoting the synthesis of hyaluronic acid; and d) reducing transepidermal water loss.

In the food composition of the present invention, the tilianin or sitologically acceptable salt thereof restores the contents of skin moisture and hyaluronic acid reduced by various external factors such as ultraviolet radiation, upregulates the expression of hyaluronic acid synthase reduced by ultraviolet radiation and reduces TEWL increased by ultraviolet radiation.

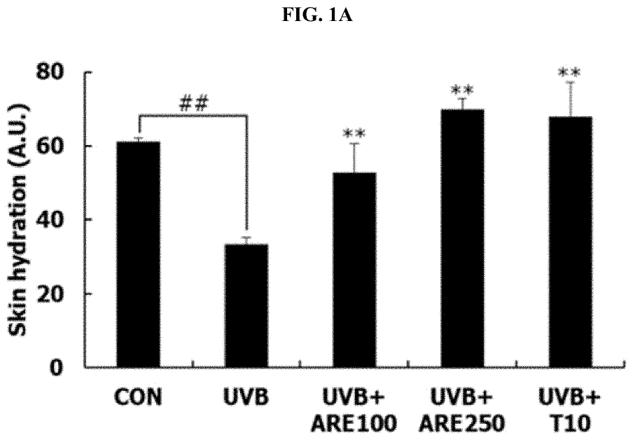

Specifically, as can be confirmed in FIG. 1, tilianin restores the moisture content in the skin reduced by UVB treatment to normal levels, thereby reducing TEWL, which represents skin barrier damage, to a significant level, and thus, it exhibits an excellent effect in enhancing skin moisture by protecting the skin barrier.

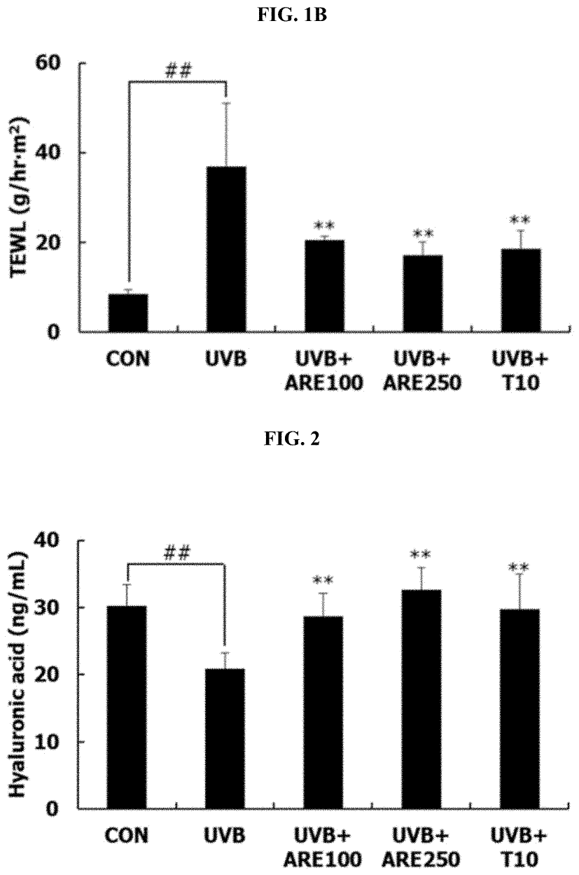

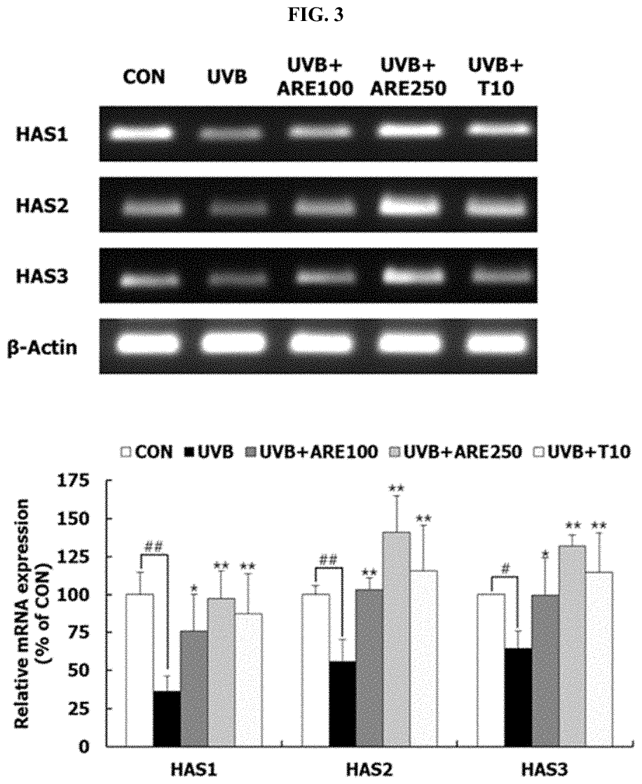

In addition, as can be confirmed in FIG. 2, tilianin significantly restores the content of hyaluronic acid in the skin reduced by UVB treatment, and as can be confirmed in FIG. 3, it significantly increases the mRNA expression levels of HAS1, HAS2 and HAS3, which are hyaluronic acid synthases, and thus, the skin moisturizing effect may be exhibited as a separate efficacy with the skin barrier protection.

As used herein, the term “sitologically acceptable salt” may be an acid addition salt formed by a free acid. Acid addition salts may be prepared by conventional methods, for example, by dissolving a compound in an excess of an aqueous acid solution and precipitating the salt using a water-miscible organic solvent such as methanol, ethanol, acetone or acetonitrile. In addition, it is possible to heat equimolar amounts of the compound and an acid or alcohol in water e.g., glycol monomethyl ether) and then evaporate the mixture to dry, or to filter the precipitated salt by suction.

As the free acid, an inorganic acid or an organic acid may be used. Non-limiting examples of the inorganic acid include hydrochloric acid, phosphoric acid, sulfuric acid, nitric acid, tartaric acid and the like, which may be used alone or in combination of two or more. Non-limiting examples of the organic acid include methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid and the like. These may be used alone or in combination of two or more.

In addition, the tilianin may be made into a pharmaceutically acceptable metal salt using a base. The alkali metal or alkaline earth metal salt may be obtained, for example, by dissolving a compound in an excess of an alkali metal hydroxide or alkaline earth metal hydroxide solution, filtering the undissolved compound salt and then evaporating and drying the filtrate. The metal salt may be prepared particularly as a sodium, potassium or calcium salt, but is not limited thereto. In addition, the corresponding silver salt may be obtained by reacting an alkali metal or alkaline earth metal salt with a suitable silver salt (e.g., silver nitrate).

Unless otherwise indicated, the salt of tilianin may include salts of both acidic or basic groups that may be present in the compound of tilianin. For example, the salt of tilianin may include sodium, calcium and potassium salts with hydroxy groups and the like, and other cosmetically acceptable salts with amino groups may include, hydrobromide, sulfate, hydrogen sulfate, phosphate, hydrogen phosphate, dihydrogen phosphate, acetate, succinate, citrate, tartrate, lactate, mandelate, methanesulfonate (mesylate), p-toluenesulfonate (tosylate) salts and the like, and it may be prepared by methods known in the art for preparing salts.

As used herein, the term “moisturizing” refers to any action that supplies moisture to the skin or blocks the evaporation of moisture to maintain the skin's flexibility and induce uniform exfoliation of dead skin cells to maintain a smooth surface, and the term “drying” “means a state in which this skin moisture is insufficient.

As used herein, the term “skin barrier strengthening” includes all of ameliorating or alleviating skin harrier damage or preventing the occurrence of skin harrier damage.

As used herein, the term “skin moisturization” includes all of ameliorating or alleviating the symptoms of dry skin, preventing the development of the symptoms of dry skin, or delaying the development of dry skin.

As used herein, the term “health functional food” includes both the meanings of “functional food” and “health food.”

As used herein, the term “functional food” is the same term as food for special healthuse (FoSHU), and means a food product with high edicinal and medical effects, which is processed to efficiently exhibit bioregulatory functions in addition to nutrition supply.

As used herein, the term “health food” refers to food having an active health maintenance or effect as compared with general food, and health supplement food refers to food for the purpose of health supplementation. In some cases, the terms of functional food, health food and health functional food are interchangeably used. The food may be prepared in various forms such as tablets, capsules, powders, granules, liquids, pills and the like.

As a specific example of such functional food, by using the composition, it is possible to produce processed food with improved storage properties while modifying to take into account the characteristics of agricultural products, livestock products or aquatic products.

The health functional food composition of the present invention may also be prepared in the form of nutritional supplements, food additives and the like, and it is intended for consumption by mammals including humans.

This type of food compositions may be prepared in various forms according to conventional methods known in the art. As general food, tilianin or a sitologically acceptable salt thereof may be added to produce beverages (including alcoholic beverages), fruit and processed food thereof (e.g., canned fruit, bottled fruit, jam, marmalade, etc), fish, meat and processed food thereof (e.g., ham, sausage, corned beef, etc.), bread and noodles (e.g., udon, soba, ramen, spaghetti, macaroni, etc.,), fruit juice, various drinks, cookies, Korean hard taffy, dairy products (e.g., butter, cheese, etc.), edible vegetable oils and fats, margarine, vegetable proteins, retort food, frozen food, various seasonings (e.g., soybean paste, soy sauce, sauce, etc.) anand the like, but is not limited thereto.

In addition, as nutritional supplements, tilianin or a sitologically acceptable salt thereof may be added to produce capsules, tablets, pills and the like, but is not limited thereto.

In addition, as health functional food, for example, tilianin or a sitologically acceptable salt thereof may be prepared in the form of tea, juice and drink and consumed by liquefying, granulating, encapsulating and powdering such that it may be ingested health drink), but is not limited thereto. Also, in order to use the tilianin or a sitologically acceptable salt thereof in the form of food additives, it may be prepared and used in the form of powder or a concentrate. In addition, the tilianin or a sitologically acceptable salt thereof may be mixed with a known active ingredient known to be effective in improving skin conditions, for example, strengthening a skin barrier, enhancing skin moisture, improving skin wrinkles or enhancing skin elasticity, and prepared in the form of a composition.

When the food composition of the present invention is used as a health beverage composition, the health beverage composition may contain various flavoring agents, natural carbohydrates or the like as additional components, as in conventional beverages. The aforementioned natural carbohydrates may be monosaccharides such as glucose and fructose; disaccharides such as maltose and sucrose; polysaccharides such as dextrin and cyclodextrin; sugar alcohols such as xylitol, sorbitol, erythritol and the like. As sweetening agents, natural sweetening agents such as thaumatin and stevia extract; synthetic sweeteners such as saccharin, aspartame and the like may be used. The proportion of such natural carbohydrates is generally about 0.01 to 0.04 g, preferably about 0.02 to 0.03 g per 100 mL, of the composition of the present invention.

Tilianin or a sitologically acceptable salt thereof may be contained as an active ingredient of a food composition for strengthening a skin barrier or moisturizing skin, and the amount thereof is preferably an amount effective to obtain the effect, for example, 0.01 to 100 wt. % based on the total weight of the entire composition, but is not particularly limited thereto.

In addition to the above, the health functional food of the present invention may contain various nutrients, vitamins, electrolytes, flavoring agents, coloring agents, pectic acid, salts of pectic acid, alginic acid, salts of alginic acid, organic acids, protective colloid thickeners, pH regulators, stabilizers, antiseptics, glycerin, alcohols. carbonating agents or the like. In addition, the health food of the present invention may contain fruit flesh for the production of natural fruit juice, fruit juice beverages, or vegetable beverages. These components may be used independently or in combination. The proportion of these additives is not critically important, but is generally chosen in a range from 0.01 to 0.1 parts by weight per 100 parts by weight of the composition of the present invention.

A second aspect of the present invention relates to a quasi-drug composition for strengthening a skin barrier or moisturizing skin, including tilianin or a pharmaceutically acceptable salt thereof.

In the quasi-drug composition of the present invention, the description of tilianin as an active ingredient and effects thereof are the same as those described in the first aspect, and thus, the description thereof is omitted.

As used herein, the term “pharmaceutically acceptable” means that it is physiologically acceptable and does not typically produce an allergic or similar reaction when administered to humans, and as the salt, an acid addition salt formed with a pharmaceutically acceptable free acid is preferred.

The pharmaceutically acceptable salt may be an acid addition salt formed with an organic or inorganic acid, and the organic acid includes, for example, formic acid, acetic acid, propionic acid, lactic acid, butyric acid, isobutyric acid, trifluoroacetic acid, malic acid, maleic acid, malonic acid, fumaric acid, succinic acid, succinic acid monoamide, glutamic acid, tartaric acid, oxalic acid, citric acid, glycolic acid, glucuronic acid, ascorbic acid, benzoic acid, phthalic acid, salicylic acid, anthranilic acid, dichloroacetic acid, aminooxyacetic acid, benzenesulfonic acid, p-toluenesulfonic acid or methanesulfonic acid. The inorganic acid includes, for example, hydrochloric acid, hydrobromic acid, sulfuric acid, phosphoric acid, nitric acid, carbonic acid or boric acid. The acid addition salt may preferably be in the form of a hydrochloride salt or an acetate salt, and more preferably, in the form of a hydrochloride salt.

In addition to the above, the additionally possible salt form includes a GABA salt, a gabapentin salt, a pregahalin salt, a nicotinate salt, an adipate salt, a hemimalonate, a cysteine salt, an acetylcysteine salt, a methionine salt, an arginine salt, a lysine salt, an ornithine salt, an aspartate salt or the like.

In addition to the above components, the quasi-drug composition of the present invention may further include a pharmaceutically acceptable carrier, an excipient or a diluent if necessary. The pharmaceutically acceptable carrier, excipient or diluent is not limited as long as it does not impair the effects of the present invention, and includes, for example, fillers, extenders, binders, wetting agents, disintegrants, surfactants, lubricants, sweetening agents, fragrances, preservatives and the like.

The term “pharmaceutically acceptable carrier” may refer to a carrier, excipient or diluent that does not interfere with the biological activity and properties of the compound being infused without stimulating the organism, and specifically, it may be a non-naturally occurring carrier. The type of the carrier that may be used in the present invention is not particularly limited, and any carrier that is generally used in this technical field and is pharmaceutically acceptable may be used. Non-limiting examples of the carrier include saline, sterile water. Ringer's solution, buffered saline, an albumin injection solution, a dextrose solution, a maltodextrin solution, glycerol, ethanol and the like. These may be used alone or in combination of two or more.

The composition including a pharmaceutically acceptable carrier may be in various oral or parenteral formulations. In the case of formulation, it is prepared using diluents or excipients such as fillers, extenders, binders, wetting agents, disintegrating agents and surfactants that are generally used. Specifically, solid preparations for oral administration include tablets, pills, powders, granules, capsules, and the like, and such solid preparations may be prepared by mixing the compound with at least one or more excipients such as starch, calcium carbonate, sucrose, lactose, gelatin or the like. In addition, lubricants such as magnesium stearate and talc may be used in addition to simple excipients. Liquid preparations for oral use include suspensions, liquid solutions, emulsions, syrups and the like, which may contain various excipients, for example, wetting agents, sweetening agents, flavoring agents, preservatives and the like, in addition to water and liquid paraffin which are simple diluents that are commonly used. Preparations for parenteral administration include sterile aqueous solutions, non-aqueous solutions, suspensions, emulsions, lyophilized formulations and suppositories. As the non-aqueous solvent and the suspending agent, propylene glycol, polyethylene glycol, vegetable oils such as olive oil, injectable esters such as ethyl oleate and the like may be used. As bases of suppositories, Witepsol, Macrogol, Tween 61, cacao butter, lauric fat, glycerogelatin and the like may be used.

The quasi-drug composition of the present invention may be exemplified by a disinfectant cleaning agent, a shower foam, an ointment liquid, a wipe, a coating agent and the like, but is not limited thereto, and the formulation method, dose, usage method, component and the like of the quasi-drug may be appropriately selected from conventional techniques known in the art.

When the tilianin or a pharmaceutically acceptable salt thereof according to the present invention is used as a quasi-drug additive, the tilianin or a pharmaceutically-acceptable salt thereof may be added as it is, or may be used in combination with other quasi-drugs or quasi-drug components, and may be suitably used according to conventional methods. The mixing amount of the active ingredient may be suitably determined according to the purpose of use.

A third aspect of the present invention relates to a composition for external use on skin for strengthening a skin barrier or moisturizing skin, including tilianin or a pharmaceutically acceptable salt thereof.

In the composition for external use on skin for strengthening a skin barrier or moisturizing skin according to the present invention, the description of the properties and effects of the tilianin or a pharmaceutically acceptable salt thereof is the same as described above, and thus, the description thereof is omitted.

Examples of the external skin preparation of the present invention include formulations in transdermal dosage forms such as lotions, ointments, gels, creams, patches or sprays. Also, in the external preparation composition of each dosage form, components other than the essential components may be appropriately selected and blended by those skilled in the art according to the dosage form of other external preparations, the purpose of use and the like without difficulty.

A fourth aspect of the present invention relates to a cosmetic composition for strengthening a skin barrier or moisturizing skin, including tilianin or a cosmetically acceptable salt thereof.

In the cosmetic composition for strengthening a skin barrier or moisturizing skin according to the present invention, the description of the properties and effects of the tilianin or a cosmetically acceptable salt thereof is the same as described above, and thus, the description thereof is omitted.

The cosmetic composition according to the present invention may be prepared in a formulation selected from the group consisting of a solution, an external ointment, cream, foam, nourishing water, softening water, a pack, soft water, an emulsion, a makeup base, an essence, a soap, a liquid detergent, a bath agent, sunscreen cream, sun oil, a suspension, an emulsion, a paste, gel, lotion, powder, soap, a surfactant-containing cleanser, oil, a powder foundation, an emulsion foundation, wax, a foundation, a patch and a spray, but is not limited thereto.

Specifically, the cosmetic composition may further include at least one substance selected from the group consisting of oil, water, a surfactant, a humectant, alcohol, a thickener, a chelating agent, a dye, a preservative and perfume, but is not limited thereto.

When the formulation of the present invention is an ointment, a paste, cream or gel, animal oil, vegetable oil, wax, paraffin, starch, tragacanth, a cellulose derivative, polyethylene glycol, silicone, bentonite, silica, talc, zinc oxide or a mixture thereof may be used.

In addition, when the formulation of the present invention is powder or a spray, lactose, talc, silica, aluminum hydroxide, calcium silicate, polyamide powder or a mixture thereof may be used, and particularly in the case of a spray, it may additionally include a propellant such as chlorofluorohydrocarbon, propane/butane or dimethyl ether.

In addition, when the formulation of the present invention is a solution or an emulsion, a solvent, a solubilizing agent or an emulsifying agent may be used, and for example, water, ethanol, isopropanol, ethyl carbonate, ethyl acetate, benzyl alcohol, benzyl benzoate, propylene glycol and 1,3-butylglycol oil may be used. In particular, cottonseed oil, peanut oil, corn germ oil, olive oil, castor oil and sesame oil, glycerol fatty ester and fatty acid ester of polyethylene glycol or sorbitan may be used.

In addition, when the formulation of the present invention is a suspension, water, a liquid diluent such as ethanol or propylene glycol, a suspending agent such as ethoxylated isostearyl alcohol, polyoxyethylene sorbitol ester and polyoxyethylene sorbitan ester, microcrystalline cellulose, aluminum, ahydroxide, bentonite, agar, tragacanth or the like may be used.

In addition, when the formulation of the present invention is a soap, alkali metal salts of fatty acid, fatty an acid hemiester salt, fatty acid protein hydrolyzate, isethionate, a lanolin derivative, aliphatic alcohol, vegetable oil, glycerol, sugar and the like may be used. The cosmetic composition of the present invention may contain commonly used adjuvants such as hydrophilic or lipophilic gelling agents, hydrophilic or lipophilic active agents, preservatives, antioxidants, solvents, fragrances, fillers, blockers, pigments, deodorants, dyes and the like.

Specifically, it is possible to provide a cosmetic composition in which the tilianin or a cosmetically acceptable salt thereof is included at 0.0001 wt. % to 20 wt. % based on the total weight of the composition.

A fifth aspect of the present invention relates to a method for strengthening a skin barrier or moisturizing skin, including administering tilianin, a sitologically acceptable salt, a pharmaceutically acceptable salt thereof or a cosmetically acceptable salt thereof to a subject in need thereof.

In the method for strengthening a skin barrier or enhancing skin moisture according to the present invention, the description of the properties and effects of the tilianin, a sitologically acceptable salt thereof, a pharmaceutically acceptable salt thereof or a cosmetically acceptable salt thereof is the same as described above, and thus, the description thereof is omitted.

As used herein, the term “administration” means introducing tilianin, a sitologically acceptable salt thereof or a pharmaceutically acceptable salt thereof to a subject in any suitable manner, or introducing the composition of the present invention including any one of the above, and the route of administration of the composition may be administered via any general route as long as it can reach the target tissue. It may be administered by intraperitoneal administration, intravenous administration, intramuscular administration, subcutaneous administration, intradermal administration, oral administration, topical administration or intranasal administration, but is not limited thereto.

Alternatively, the composition may be applied by topical application.

In the present invention, since tilianin exhibits skin barrier strengthening and/or skin moisturizing effects through oral administration, the route of administration of the composition may preferably be oral administration.

The composition of the present invention may be administered daily or intermittently, and the number of administrations per day may be administered once or in 2 to 3 divided doses. In addition, the composition of the present invention may be used alone or in combination with other drug treatments for strengthening a skin barrier and/or moisturizing skin. In consideration of all of the above factors, it is important to administer an amount that may obtain the maximum effect with a minimum amount without side effects, and it may be easily determined by those skilled in the art.

As used herein, the term “subject” refers to a variety of mammals, including humans, in which skin barrier damage or dry skin has occurred or may occur, but is not limited thereto.

The preferred dose of the composition of the present invention varies depending on the condition and body weight of a subject, the degree of fatigue, the form of the preparation of an active ingredient and the like, but it may be appropriately selected to achieve the intended skin barrier strengthening or skin moisture enhancing effect.

As used herein. the term “effective amount” means an amount of tilianin, a sitologically acceptable salt thereof or a pharmaceutically acceptable salt thereof, which is sufficient to restore the contents of moisture and hyaluronic acid in the skin reduced due to external stimuli and to upregulate HAS, thereby exhibiting excellent skin barrier strengthening and/or skin moisturizing effects. For the desired effect, the tilianin of the present invention, a sitologically acceptable salt thereof or a pharmaceutically acceptable salt thereof may be administered once or repeatedly administered several times at regular time intervals.

In the method for strengthening a skin barrier or enhancing skin moisture according to the present invention, the composition including the tilianin, a sitologically acceptable salt thereof or a pharmaceutically acceptable salt thereof may he administered to a subject by various routes, and preferably, it may be orally administered. It may also be used alone or in combination with other methods for strengthening a skin barrier or enhancing skin moisture.

A sixth aspect of the present invention relates to a use of tilianin, a sitologically acceptable salt thereof, a pharmaceutically acceptable salt thereof or a cosmetically acceptable salt thereof in the preparation of food, medicine or cosmetic products for strengthening a skin barrier or enhancing skin moisture.

In the use of the present invention, the food may be health functional food, and the description of the properties and effects of the tilianin, a sitologically acceptable salt thereof, a pharmaceutically acceptable salt thereof or a cosmetically acceptable salt thereof is the same as described above, and thus, the description thereof is omitted.

A seventh aspect of the present invention relates to a composition for preventing, ameliorating or treating skin disease, including tilianin.

In this regard, the present invention specifically provides a pharmaceutical composition for preventing or treating skin disease, including tilianin or a pharmaceutically acceptable salt thereof; a health functional food composition for preventing or ameliorating skin disease, including tilianin or a sitologically acceptable salt thereof; a composition for external use on skin for preventing or treating skin disease, including tilianin or a pharmaceutically acceptable salt thereof; and/or a cosmetic composition for preventing or ameliorating skin disease, including tilianin or a cosmetically acceptable salt thereof.

The description of the properties and effects of the tilianin, a sitologically acceptable salt thereof or a pharmaceutically acceptable salt thereof is the same as described above, and thus, the description thereof is omitted.

In the composition for preventing, ameliorating or treating skin disease according to the present invention, the skin disease may be caused by skin barrier damage or dry skin, and for example, it may be one or more selected from the group consisting of dry skin, eczema, psoriasis, atopic dermatitis, ichthyosis and pemphigus, but is not limited thereto. In addition, any disease in which the skin moisture level is reduced as a result of the corresponding disease may also be included without limitation.

In an exemplary embodiment of the present invention, as shown in FIG. 1A and FIG. 1B, it was confirmed that tilianin restored the moisture content in the skin reduced by UVB treatment to normal levels, thereby significantly reducing TEWL, which represents skin barrier damage.

In addition, as can be confirmed in FIG. 2, tilianin significantly restores the content of hyaluronic acid in the skin reduced by UVB treatment, and as can be confirmed in FIG. 3, it significantly increases the mRNA expression levels of HAS 1, HAS2 and HAS 3, which are hyaluronic acid synthases, such that the skin moisturizing effect may be exhibited as a separate efficacy from the skin barrier protection.

Accordingly, through the effects of tilianin for increasing the concentration of hyaluronic acid in the skin, reducing the degree of transepidermal water loss (amount of transepidermal water loss) and increasing the moisture content, it was confirmed that it may be applied to the prevention, amelioration or treatment of various skin diseases caused by skin barrier damage or dry skin.

The pharmaceutical composition of the present invention may further include a pharmaceutically acceptable carrier. The pharmaceutically acceptable carrier may further include, for example, a carrier for oral administration or a carrier for parenteral administration. Carriers for oral administration may include lactose, starch, cellulose derivatives, magnesium stearate, stearic acid and the like. Carriers for parenteral administration may include water, suitable oil, saline, aqueous glucose, glycol and the like. In addition, it may further include stabilizers and preservatives. Suitable stabilizers include antioxidants such as sodium bisulfite, sodium sulfite or ascorbic acid. Suitable preservatives include benzalkonium chloride, methyl- or propyl-paraben and chlorobutanol. As other pharmaceutically acceptable carriers, reference may be made to those described in the following document (Remington's Pharmaceutical Sciences, 19th ed., Mack Publishing Company, Easton, Pa., 1995).

The pharmaceutical composition of the present invention may be administered in any manner to mammals, including humans. For example, it may be administered orally or parenterally, and parenteral administration may be intravenous, intramuscular, intraarterial, intramedullary, intrathecal, intracardiac, transdermal, subcutaneous, intraperitoneal, intranasal, enteral, topical, sublingual or rectal administration, but is not limited thereto.

The pharmaceutical composition of the present invention may be formulated into a preparation for oral administration or parenteral administration according to the administration route as described above. When formulated, it may be formulated with one or more buffering agents (e.g., saline or PBS), carbohydrates (e.g., glucose, mannose, sucrose, or dextran, etc.), antioxidants, bacteriostats, chelating agents (e.g., EDTA or glutathione), fillers, extenders, binders, adjuvants (e.g., aluminum hydroxide), suspending agents, thickening agents, wetting agents, disintegrating agents or surfactants, diluents or excipients.

Solid preparations for oral administration include tablets, pills, powders, granules, liquids, gels, syrups, slurries, suspensions or capsules, and such solid preparations may be prepared by mixing the pharmaceutical composition of the present invention with at least one or more excipients such as starch (including corn starch, wheat starch, rice starch, potato starch, etc.), calcium carbonate, sucrose, lactose, dextrose, sorbitol, mannitol, xylitol, erythritol, maltitol, cellulose, methyl cellulose, sodium carboxymethylcellulose, hydroxypropylmethyl-cellulose, gelatin or the like. For example, tablets or sugar tablets may be obtained. by blending the active ingredient with a solid. excipient, grinding it, and processing it into a granule mixture after adding suitable adjuvants.

Lubricants such as magnesium stearate talc are also used in addition to simple excipients. Liquid formulations for oral use include suspensions, solutions, emulsions, syrups or the like, and various excipients, for example, wetting agents, sweetening agents, fragrances, preservatives or the like may be included, in addition to water or liquid paraffin which are commonly used simple diluents.

In addition, in some cases, cross-linked polyvinylpyrrolidone, agar, alginic acid, sodium alginate or the like may be added as a disintegrant, and an anti-aggregating agent, a lubricant, a wetting agent, a flavoring agent, an emulsifying agent, a preservative agent and the like may be further included.

For parenteral administration, the pharmaceutical composition of the present invention may be formulated in the form of an injection, a transdermal administration and a nasal inhalation together with suitable parenteral carriers according to methods known in the art. The injection must be sterilized and protected from contamination of microorganisms such as bacteria and fungi. Examples of suitable carriers for injection may be solvents or dispersion media including water, ethanol, polyols (e.g., glycerol, propylene glycol, liquid polyethylene glycol, etc.), mixtures thereof and/or vegetable oils, but are not limited thereto. More preferably, as suitable carriers, Hanks' solution, Ringer's solution, phosphate buffered saline (PBS) containing triethanolamine or isotonic solutions such as sterile water for injection, 10% ethanol, 40% propylene glycol, 5% dextrose and the like may be used. In order to protect the injection from microbial contamination, various antibacterial and antifungal agents such as paraben, chlorobutanol, phenol, sorbic acid, thimerosal and the like may be further included. In addition, in most cases, the injection may further include isotonic agents such as sugar or sodium chloride.

In the case of transdermal administration, forms such as an ointment, cream, lotion, gel, an external solution, a paste preparation, a liniment, aerosol and the like are included. In the above, the term ‘transdermal administration’ means that an effective amount of the active ingredient contained in the pharmaceutical composition is delivered into the skin by topically administering the pharmaceutical composition to the skin.

In the case of inhalation administration, the compound used in accordance with the present invention may be conveniently delivered in the form of an aerosol spray from s pressurized pack or a nebulizer, with the use of a suitable propellant such as dichlorofluoromethane, trichlorofluoromethane, dichlorotetrafluoroethane, carbon dioxide or other suitable gas. In the case of a pressurized aerosol, the dosage unit may be determined by providing a valve to deliver a measured amount. For example, gelatin capsules and cartridges used in inhalers or insufflators may be formulated to contain a powder mixture of the compound and a suitable powder base such as lactose or starch. Formulations for parenteral administration are described in the following document which is a commonly known prescription in all of pharmaceutical chemistry (Remington's Pharmaceutical Science, 15th Edition, 1975. Mack Publishing Company, Easton, Pa. 18042, Chapter 87: Blaug, Seymour).

The pharmaceutical composition of the present invention may provide a desirable skin disease prevention, amelioration or therapeutic effect when it includes an effective amount of tilianin or a pharmaceutically acceptable salt thereof. As used herein, the term “effective amount” refers to an amount that exhibits a response that is greater than that of a negative control, and refers to a sufficient amount to prevent, ameliorate or treat various skin diseases caused by skin barrier damage or dry skin, through the effects of increasing the concentration of hyaluronic acid in the skin, decreasing the degree of transepidermal water loss (transepidermal water loss) and increasing the moisture content. The pharmaceutical composition of the present invention may include 0.01 to 99.9% of tilianin or a pharmaceutically acceptable salt thereof, with the remainder being a pharmaceutically acceptable carrier. An effective amount of tilianin or a pharmaceutically acceptable salt thereof included in the pharmaceutical composition of the present invention may vary depending on the form in which the composition is to be manufactured and the like.

The total effective amount of the pharmaceutical composition of the present invention may be administered to a patient in a single dose, or may also be administered by a fractionated treatment protocol in which multiple doses are administered over a long period of time. The pharmaceutical composition of the present invention may have a varied content of the active ingredient depending on the condition of the patient. For example, it may be administered in a single dose to several divided doses such that it is preferably administered in an amount of 0.001 to 100 mg/kg of body weight per day or more preferably 0.01 to 10 mg/kg of body weight per day, based on tilianin or a pharmaceutical acceptable salt thereof. However, for the dose of the tilianin or a pharmaceutically acceptable salt thereof, the effective dose for a patient is determined by considering various factors such as the patient's age, body weight, health status, gender, severity of the disease, diet and excretion rate, as well as the route of administration and number of treatments of the pharmaceutical composition, and thus, one of ordinary skill in the art will be able to determine the appropriate effective dose of tilianin or a pharmaceutically acceptable salt thereof according to the particular use for preventing, ameliorating or treating various skin diseases caused by skin barrier damage or dry skin in view of the above factors. The pharmaceutical composition according to the present invention is not particularly limited in its formulation, administration route and administration method as long as it shows the effects of the present invention.

An eighth aspect of the present invention relates to a method for preventing, ameliorating or treating skin disease, including administering tilianin, a sitologically acceptable salt thereof or pharmaceutically acceptable salt thereof to a subject in need thereof.

In the method for preventing, ameliorating or treating skin disease according to the present invention, the description of the properties and effects of tilianin, a sitologically acceptable salt thereof or a pharmaceutically acceptable salt thereof is the same as described above, and thus, the description thereof is omitted.

In the method for preventing, ameliorating or treating skin disease according to the present invention, the type of the skin disease, the condition of the subject, the administration method, the administration dose and the like are the same as those described in the above-mentioned method for strengthening a skin barrier or enhancing skin moisture in the fourth aspect, and thus, the description thereof is omitted.

Finally, a ninth aspect of the present invention relates to a use of tilianin, a sitologically acceptable salt thereof, a pharmaceutically acceptable salt thereof or a cosmetically acceptable salt thereof in the preparation of food, medicine or cosmetic products for preventing, ameliorating or treating skin disease.

Similarly, in the above-mentioned use, the description of the properties and effects of tilianin, a sitologically acceptable salt thereof, a pharmaceutically acceptable salt thereof or a cosmetically acceptable salt thereof, and the type of the skin disease is the same as described above, and thus, the description thereof is omitted.

Hereinafter, the present invention will be described in more detail through examples. These examples are only for illustrating the present invention, and it will be apparent to those of ordinary skill in the art that the scope of the present invention is not to be construed as being limited by these examples.

### EXAMPLES

### Example 1: Standardization of Agastache rugosa Extract and Analysis of Tilianin

1-1. Preparation of Agastache rugosa Extract

The aerial part of Agastache rugosa (A. rugosa) was supplied from Nutribiotech. The aerial part was ground with a mixer and then extracted with water at 95° C. for 4 hours using a rotating impeller (MS3040, Misung Co.). The solvent was completely removed from the extract by a vacuum rotary evaporator (Heidolph Instruments GmbH & Co. KG) to obtain ARE. The final yield of ARE was 10.0% (w/w).

1-2. Standardization of Agastache rugosa Extract and Analysis of Tilianin

In order to standardize ARE via tilianin, the identification and determination of the content of tilianin in the ARE were performed by high-performance liquid chromatography via the Agilent Technologies 1260 Infinity II HPLC system (Agilent Techniques) equipped with a Sunfire C18 column (150×4.6 mm, 5 μm id; Waters). The location of tilianin in the chromatographic peak of the ARE was determined by comparison with the retention time of pure tilianin (Chengdu Biopurify Phytochemicals LTD.). A standard curve between the measured values was generated by preparing tilianin for each concentration, and the content of tilianin in the ARE was determined based on the standard curve. The content of tilianin in the ARE was 0.75% (w/w).

### Example 2: Confirmation of Effects of Protecting Skin Barrier and Enhancing Skin Moisture

2-1. Animal Breeding and Oral Administration

Five-week-old female hairless mice (SKFI-1) were purchased from Orient Bio (Seongnam, Korea) as experimental animals. Diets of feed and water were freely given, and the animals were bred at the Yonsei Laboratory Animal Research Center (YLARC, Seoul, Korea) under conditions of 25° C. and 50 to 60% humidity for 12 hours. The hairless mice were randomly assigned to the following five groups: (i) a control group, (ii) a UVB-irradiated group (UVB), (iii) a UVB+100 mg/kg/day ARE-administered group (ARE100), (iv) a UVB+250 mg/Kg/day ARE-administered group (ARE250) and (v) UVB+10 mg/kg/week tilianin-administered group (T10). After one week of acclimatization, oral administrations of ARE and tilianin were performed daily for 12 weeks. In this case, the hairless mice belonging to the CON group and the UVB groups were orally administered with saline instead of the samples. The hairless mice of the remaining groups except the CON group were irradiated with UVB, and the UVB irradiation was carried out using UV crosslinker CL-1000M (UVP) toward the back 3 times per week. The irradiation dose was started at 75 mJ/cm², and the irradiation dose was increased by 1 minimal erythema dose (MED) per week and finally maintained at an intensity of 3 MED until the end of oral administration. At the end of the oral administration period, 325 mg/kg tribromoethanol (Sigma Aldrich) was injected intraperitoneally to induce anesthesia, and the animals were sacrificed via cardiac hemorrhage. The skin was removed from the dorsal section of the hairless mice and rapidly frozen in liquid nitrogen for molecular biology analysis and then stored at −70° C., or some were fixed with 10% formaldehyde (Junsei Chemical Co., Ltd., Tokyo, Japan) for histopathological analysis.

The animal experiment was carried out with the approval of the Institute of Animal Care and Use Committee (IACUC) of Yonsei University (Approval No.: IACUC-A-201803-203-02).

2-2. Measurement of Moisture Content and Transepidermal Water Loss (Amount of Transepidermal Water Loss)

Before anesthetizing the hairless mice, the moisture content and the degree of transepidermal water loss (TEWL) were measured on the dorsal section of the hairless mice. Moisture content was measured using CORNEOMETER® 825 (CK Electronics GmbH) at a pressure of 1.1 to 1.5 N after contacting a probe to the skin surface. The TEWL which represents the amount of the evaporation of transepidermal water in the process of damage and recovery of a skin barrier was measured under conditions of 20 to 25° C. and 40 to 60% humidity using TEWAMETER® TM300 (CK Electronics GmbH) equipped with MULTI PROBE ADAPTER® MPA5 (CK Electronics GmbH).

As can be confirmed in FIG. 1A, the moisture content in the skin was reduced by 45.38% in the UVB groups compared to the CON group, but it was significantly restored by the treatment with ARE in a concentration-dependent matter. In particular, the moisture contents in the ARE250 and T10 groups were restored to normal levels. As the moisture content in the skin was restored, additionally, TEWL which represents damage to the skin barrier was measured. As can be confirmed in FIG. 1B, as UVB was treated, TEWL was increased compared to the CON group and it was decreased to a significant level by the treatments with ARE and tilianin.

These results indicate that the ARE and tilianin Unproved skin moisture by protecting the skin barrier from UVB.

### Example 3: Confirmation of the Effect of Increasing the Concentration of Hyaluronic Acid

Hyaluronic acid (HA), which is a polymer composed of repetitive disaccharides, is one of the key components of the dermal extracellular matrix (ECM), and is synthesized from nucleotide sugars activated by HA synthase present in three isoforms in the plasma membrane. The structural function of HA to provide skin volume and elasticity by moisture retention is related to its molecular structure. In addition, HA initiates receptor signaling and creates a skin extracellular condition that supports physicochemical properties. Therefore, in this Example, the change in the concentration of hyaluronic acid in the skin following the administration of tilianin was evaluated in order to confirm the skin moisturizing effect of tilianin.

Specifically, the skin tissues of the hairless mice of each group in Example 2-1 were homogenized with NP40 lysis buffer (Elpis Biotech, Daejeon, Korea). The concentration of hyaluronic acid in the homogenate was determined using the hyaluronic acid quantikine ELISA Kit (R&D Systems, Minneapolis, Minn., USA) according to the manufacturer's protocol. Absorbance was read at 450 nm using the VERSAMAX™ variable microplate reader (Molecular Devices, Inc., Sunnyvale, Calif., USA).

As can be confirmed in FIG. 2, the content of hyaluronic acid was reduced by 31.02% in the UVB-treated group compared to the control group, and it was increased by 37.43%, 56.19% and 42.26% in the UVB+ARE100 group, the UVB+ARE250 group and the UVB+T10 group, respectively, compared to the UVB-treated group.

### Example 4: Confirmation of the Effect of Increasing the Expression of Hyaluronic Acid Synthase

Hyaluronic acid synthase (HAS) is classified into three different isoforms, HAS1, HAS2 and HAS3, which are key proteins responsible for HA synthesis. Accordingly, in this Example, the change in the expression of HAS mRNA according to the administration of tilianin was evaluated as another method for confirming the skin moisturizing effect of tilianin.

Specifically, mRNA was isolated from the skin tissues of each group in Example 2-1 using RNAISO™ Plus (Takara). The mRNA was quantified using the NANODROP™ 1000 spectrophotometer (Thermo Fisher Scientific Inc.). An equal amount of mRNA was added to Reverse Transcriptase Premix (Elpis Biotech, Inc.), and cDNA was synthesized using Gene Amp PCR System 2700 (Applied Biosystems) under conditions of 42° C. for 55 minutes and 70° C. for 15 minutes. The synthesized cDNA and primers for each target gene were placed in HIPI™ PCR PREMIX (Elpis Biotech, Inc.), and PCR was performed using Gene Amp PCR System 2700 (Applied Biosystems). The nucleotide sequences of the primers are shown in Table 1, and PCR was carried out by repeating 35 to 45 cycles of the procedure of initial denaturation for 5 minutes at 94° C., followed by denaturation for 30 seconds at 98° C., annealing for 1 minute at 56° C. and extension for 1 minute at 72° C., followed by final amplification for 5 minutes at 72° C. The PCR products were stained with a loading star (DyneBio) and electrophoresed on a 1.5% agarose gel. The band expression level of each target gene was confirmed using the G:BOX image analysis system (Syngene). β-actin was used as an internal control.

TABLE 1

| | | | SEQ ID |
|---|---|---|---|
| Gene | Direction | Sequence (5’-3’) | NO: |
| HAS1 | Forward | CCGGAAGAAACTGGCTGCTA | 1 |
| | Reverse | ACTTGGACACGGTAACCACC | 2 |
| HAS2 | Forward | GTTAGTGTCTGGGTTCGCCA | 3 |
| | Reverse | GCTCTTTTTGCTTCGCCACA | 4 |
| HAS3 | Forward | TCACATATGGCTCTCATCCTGC | 5 |
| | Reverse | CCTTGGAGAAGCCACTGAGAAT | 6 |
| β- | Forward | GAAGGAGATTACTGCTCTGGCTC | 7 |
| actin | Reverse | CTCAGTAACAGTCCGCCTAGAA | 8 |

As shown in FIG. 3, it was confirmed that the mRNA expression levels of HAS1, HAS2 and HAS3, which are hyaluronic acid synthetases, were significantly increased when the Agastache rugosa extract or tilianin was orally administered, compared to the UVB-treated group.

Based on the above results, it was confirmed that tilianin exhibits excellent skin barrier strengthening and/or skin moisturizing effects by increasing the contents of moisture and hyaluronic acid in the skin, upregulating HAS and reducing the degree of transepidermal water loss (transepidermal water loss).

1. A method for strengthening a skin barrier or enhancing skin moisture, comprising administering a composition comprising tilianin, a sitologically acceptable salt thereof or a pharmaceutically acceptable salt thereof to a subject in need thereof.

2. The method of claim 1, wherein the tilianin is derived from an Agastache rugosa extract.

3. The method of claim 1, wherein the tilianin, sitologically acceptable salt thereof or pharmaceutically acceptable salt thereof is comprised at 0.0001 to 20 wt. % based on the total weight of the composition.

4. The method of claim 1, wherein the tilianin exhibits an effect of any one or more of the following a) to d):
a) increasing the content of moisture in the skin;
b) increasing the content of hyaluronic acid in the skin;
c) promoting the synthesis of hyaluronic acid; and
d) reducing transepidermal water loss.

5. The method of claim 1, wherein the composition is food.

6. The method of claim 5, wherein the food composition is a health functional food composition.

7. The method of claim 1, wherein the composition is a pharmaceutical composition or a quasi-drug composition.

8. The method of claim 1, wherein the composition is prepared in tablets, capsules, powders, granules, liquids or pills.

9. A method for preventing, ameliorating or treating skin disease, comprising administering a composition comprising tilianin, a sitologically acceptable salt thereof or a pharmaceutically acceptable salt thereof to a subject in need thereof,
wherein the skin disease is caused by damage to a skin barrier or dry skin.

10. The method of claim 9, wherein the tilianin is derived from an Agastache rugosa extract.

11. The method of claim 9, wherein the skin disease caused by damage to the skin barrier or dry skin is any one or more selected from the group consisting of dry skin, eczema, psoriasis, atopic dermatitis, ichthyosis and pemphigus.

12. The method of claim 9, wherein the tilianin exhibits an effect of any one or more of the following a) to d):
a) increasing the content of moisture in the skin;
b) increasing the content of hyaluronic acid in the skin;
c) promoting the synthesis of hyaluronic acid; and
d) reducing transepidermal water loss.

13. The method of claim 9, wherein the composition is food.

14. The method of claim 13, wherein the food composition is a health functional food composition.

15. The method of claim 9, wherein the composition is a pharmaceutical composition or a quasi-drug composition.

16. The method of claim 9, wherein the composition is prepared in tablets, capsules, powders, granules, liquids or pills.


SEQUENCE LISTING (8 sequences)

SEQ ID NO: 1 - DNA, 20 bp, Artificial
  HAS1 forward primer
ccggaagaaactggctgcta

SEQ ID NO: 2 - DNA, 20 bp, Artificial
  HAS1 reverse primer
acttggacacggtaaccacc

SEQ ID NO: 3 - DNA, 20 bp, Artificial
  HAS2 forward primer
gttagtgtctgggttcgcca

SEQ ID NO: 4 - DNA, 20 bp, Artificial
  HAS2 reverse primer
gctctttttgcttcgccaca

SEQ ID NO: 5 - DNA, 22 bp, Artificial
  HAS3 forward primer
tcacatatggctctcatcctgc

SEQ ID NO: 6 - DNA, 22 bp, Artificial
  HAS3 reverse primer
ccttggagaagccactgagaat

SEQ ID NO: 7 - DNA, 23 bp, Artificial
  Beta-actin forward primer
gaaggagattactgctctggctc

SEQ ID NO: 8 - DNA, 22 bp, Artificial
  Beta-actin reverse primer
ctcagtaacagtccgcctagaa